(19) United States
(12) Patent Application Publication
(10) Pub. No.: US 2014/0363806 A1
(43) Pub. Date: Dec. 11, 2014

(54) BIO-ELECTRO REACTORS WITH REAL-TIME ADJUSTABLE ELECTRIC PARAMETERS AND SEQUENCING PROGRAMMABLE POWER SUPPLIES

(71) Applicant: LOGOS TECHNOLOGIES, INC., Durham, NC (US)

(72) Inventors: Claudio Fillipone, College Park, MD (US); Daniel Pryor Maceachran, Washington, DC (US); Gregg Anthony Deluga, Los Angeles, CA (US)

(21) Appl. No.: 14/370,310

(22) PCT Filed: Feb. 22, 2013
(86) PCT No.: PCT/US2013/027486

§ 371 Date: Jul. 2, 2014

Related U.S. Application Data
(60) Provisional application No. 61/602,896, filed on Feb. 24, 2012.

(51) Int. Cl. C12M 1/34; C12N 1/20; C25B 1/02

(52) U.S. Cl. CPC C12M 41/34; C25B 1/02; C12N 1/20
USPC 435/3; 435/286.1

(57) ABSTRACT

Bio-electro reactors with real-time adjustable electric parameters and sequencing programmable power supplies are disclosed. According to an aspect, a bio-electro reactor for control of electrolysis gases bubbles within a biologically-active substance includes a vessel defining an interior for holding a biologically-active substance. The bio-electro reactor also includes electrodes positioned to be electrically coupled with at least a portion of the biologically-active substance. Further, the bio-electro reactor includes an electric source configured to apply voltage across the electrodes. The bio-electro reactor also includes an electrical controller configured to determine an electrical impedance at one or more of the electrodes for use in controlling electrolysis gases bubbles within the biologically active substance.

### CROSS REFERENCE TO RELATED APPLICATION

This application claims priority to U.S. Provisional Application No. 61/602,896 filed on Feb. 23, 2012, the content of which is hereby incorporated by reference in its entirety.

### TECHNICAL FIELD

The present disclosure relates to bio-electro reactors. More particularly, the present disclosure relates to bio-electro reactors with real-time adjustable electric parameters and sequencing programmable power supplies.

### BACKGROUND

A fermentor is a machine that maintains optimal conditions for the growth of microbes. These machines can be used in large-scale fermentation and in commercial production of value added compounds. Fermentation processes are commonly employed for the preparation of chemical compounds, such as amino acids or carboxylic acids. In practice, it can be difficult to operate such processes in an economically feasible way, because the costs of separating the fermentation products can be high.

Fermentors may use electrolysis, a technique of using an electric current to drive an otherwise non-spontaneous chemical reaction. In an example, hydrogen may be one of the electrolysis gases, and its production may be obtained through large-scale high temperature systems, stored and transported so that it can be fed to fermentor- or culture-reactors that require hydrogen as a feedstock. The hydrogen produced outside of the fermentor can then be fed to the fermentor by bubbling it through a sparging system. Although large scale hydrogen production can be executed at relatively low cost, the costs associated with storage, transportation, and general handling in compliance with safety provisions may be prohibitively high. Furthermore, current fermentors use either electromagenetic or mechanical stirring devices and require the use of some form of temperature regulation. The use of submersible electrodes within the growth medium and their continual electrical switching can allow for mixing of the fermentation medium and potentially temperature regulation for optimal growth of a given microbe. Fermentor reactors that combine cultivation of organisms and electrical generation of gas are described by E. Schuster and H. G. Schlegel (1967) “Chemolithotropic Growth of Hydrogenomonas H16 Using Electrolytic Production of Hydrogen and Oxygen in a Chemostat” and by by \check{R}i\check{c}ica in 1966, the disclosure of which is incorporated herein by reference, utilizing a retrofitted chemostat—a fermenter reactor to which fresh medium is continuously added, while culture medium is continuously removed to keep the culture volume constant. Both of these prior art examples represent relatively crude designs wherein electrode geometry, proximity, number and the control thereof are not optimized.

In view of the foregoing, it is desired to provide improved fermentors and fermentation techniques.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Bio-electro reactors with real-time adjustable electric parameters and sequencing programmable power supplies are disclosed. According to an aspect, a bio-electro reactor for control of electrolysis gases bubbles within a biologically-active substance includes a vessel defining an interior for holding a biologically-active substance. The bio-electro reactor also includes electrodes positioned to be electrically coupled with at least a portion of the biologically-active substance. Further, the bio-electro reactor includes an electric source configured to apply voltage across the electrodes. The bio-electro reactor also includes an electrical controller configured to determine an electrical impedance at one or more of the electrodes for use in controlling electrolysis gases bubbles within the biologically active substance.

According to another aspect, a bio-electro reactor for growth of microbes for production of chemicals via control of electrolysis gases bubbles within a biologically-active substance includes a vessel defining an interior for holding a biologically-active substance. The bio-electro reactor also includes electrodes positioned to be electrically coupled with at least a portion of the biologically-active substance. Further, the bio-electro reactor includes an electric source configured to apply voltage across the plurality of electrodes. The bio-electro reactor also includes an electrical controller configured to determine an electrical impedance at one or more of the electrodes for use in controlling electrolysis gases bubbles within the biologically active substance.

An objective is to provide a technique to program or control the distribution of electrical parameters (e.g., current, voltage, and frequency) provided to multiple independent electrodes of a fermentor. Further, it is an object to control the sequencing of these parameters. Additionally, it is an object to provide the programmed distribution of electrical parameters to the electrodes for matching the electrical load represented by the solution media and transfer electrolysis energy from the power supplies to the fermentor media while adjusting the electrolysis gas production rate, and for controlling the buoyancy of electrolysis gas bubbles. Gas bubbles buoyancy can be controlled so as to increase the gas residence time within the solution, and to ensure no or little hydrogen gas arrives at the surface of the solution. Through the regulation of electrical input the generation of hydrogen may be controlled to match that of the consumption of hydrogen gas. This will improve overall efficiency of the device as well as minimize the presence of highly reactive hydrogen and oxygen gas in the headspace.

Another objective is to provide accurate and adjustable spatial electrode distribution so as to generate uniform electric fields across the solution media while activating and deactivating each independent electrode so as to generate a controlled recirculation flow that does not require traditional power consuming stirring mechanisms.

By controlling the electrolysis gas production rate and bubble size, fermentors and techniques disclosed herein may provide for enhancement of electrolysis gas dissolution in the solution media. For application targeting microbe growth, the electrolysis gases dissolved can be used as feedstock for growth of microbes. For example, selected microbes may utilize hydrogen, carbon dioxide, and small amounts of oxygen. Accordingly, by use of fermentors and techniques disclosed herein, microbes may be efficiently engineered to produce a variety of molecules, including but not limited to methyl ketones (See e.g., PCT international application PCT/US2012/064216). By producing only the amount of gases necessary for growth of the microbes, operation of a fermentor may be safer. If excess hydrogen is produced, the headspace of the fermentor can contain a flammable mixture of oxygen and hydrogen, thereby requiring costly safety mechanism and increasing the hazard risk to operators.

The aforementioned objects and others are provided by the embodiments and examples disclosed herein.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. In the drawings:

FIG. 1 is a diagram of an example bio-electro reactor in accordance with embodiments of the present disclosure;

FIG. 2 depicts an example method of forcing flow circulation with a fermentor vessel in accordance with embodiments of the present disclosure;

FIG. 3A is a perspective view of assembled components of a fermentor vessel, electrodes, and other assembly components in accordance with embodiments of the present disclosure;

FIG. 3B is another perspective view of the assembled component shown in FIG. 3A along with another annular electrode and its associated assembly components;

FIG. 4 is a schematic illustration of the assembly shown in FIG. 3B with an extended distance between the two annular electrode;

FIG. 5 is a perspective view of the assembled component shown in FIG. 4 along with other electrodes and their associated assembly components;

FIG. 6 is a side view of a bio-reactor vessel and a top flange; and

FIGS. 7A, 7B, and 7C are perspective views showing sequential activation and deactivation of the annular electrodes to induce flow circulation patterns in accordance with embodiments of the present disclosure.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

In accordance with embodiments of the present disclosure, reactors disclosed herein can integrate features of a biological fermentation device and can supply electrical current to the system via an electronic controller to generate electrolysis gases. Further, reactors disclosed herein can generate hydrolysis gases including, for example, but not limited to, hydrogen, oxygen, and the like. In an example, a reactor may include a fermentor, submersible electrodes, an electric source, and an electrical controller (or control unit). The electrical controller is configured to monitor or determine electrical impedance to allow for the control of electrolysis gas bubble size generated from the submersed electrodes. The ability to regulate bubble size and hence gas buoyancy within the fermentor can allow for increased or decreased residence time of the gases within the growth medium. Increasing the residence time of the gases within the reactor can result in increased efficiency of the system. Gas evolution rate can be customized or adjusted. For applications targeting microbe growth, gas evolution rates can meet the needs of a given microbe and can be varied over the course of a single fermentation resulting in decreased gas waste.

An example advantage of bio-electro reactors and techniques disclosed herein is provision of the ability to supply electric current to the solution media and generate electrolysis gases in situ and in quantities that do not require safety features normally required for handling flammable electrolysis gases (i.e., hydrogen). Example electrodes utilized in bio-electro reactors disclosed herein include interchangeable anodes that may be shaped in various forms, including, for example, rings or annular bands at least partially or entirely surrounding a central cathode, thereby forming a concentric electrode configuration. Such a configuration can mitigate the problem of poor distribution of electrolysis gases generating at the surfaces of ring/annular electrodes or electrodes with other shapes. Other example electrode configurations may include electrodes formed by screens concentrically surrounding a central electrode. In another example, rectangular and other geometries involving electrode plates positioned so as to form a variable plate capacitor may be utilized.

In accordance with embodiments, an electrical controller of a bio-electro reactor may control sampling of electrical parameters characterizing the solution media and, accordingly, sequence the independent activation or deactivation of the electrodes so as to control the electrolysis gas generation rate, the gas bubble size, and the electrostatically driven flow of the electrolysis-gas-enriched solution toward desired locations within the vessel. For example, the bubbles may be controlled to move against the combined effect of gravity and electrolysis gas bubble buoyancy, without the need for mechanical stirrers.

Bio-electro-reactor disclosed herein may operate to adjust electric parameters supplied to two or more independent electrodes. An electrical controller may control an electrical source to apply voltage across the electrodes. As an example, the electrodes may be driven by multiplexed programmable power supplies configured to match the load impedance formed by the bio-electro-reactor solution media surrounding the independent electrodes and generate selected feedstock gases via low temperature electrolysis and feed said gases to microbes without harming the microbes through electric current sensing and limiting features integrated with the programmable power supplies.

In accordance with embodiments, bio-electro reactors disclosed herein may implement controls to distribute electric current flowing via multiple independent electrodes with programmable current, voltage, and frequency so as to match the electrical load represented by the solution media and transfer electrical energy into the solution media by use of programmable power supplies.

In accordance with embodiments, multiple independent electrodes may be configured so as to allow an operator to accurately, spatially distribute the electrodes within the media to ensure uniform electric fields across the media and within desired areas of the solution media.

In accordance with embodiments, an electrical controller may control programmable power supplies to activate and deactivate electrodes according to pre-programmed sequences for controlling the electrolysis gas production rate and to induce media recirculation flows that do not require additional stirring mechanisms. Additionally, by activating and deactivating the multiple independent electrodes at desired conditions, the electrodes may be electrically reset so as to represent their full electric potential to the media without saturating the electrode surfaces without requiring electronic quenching features.

As the programmable power supplies may distribute electric current to the independent electrodes at selected frequencies, the impedance represented by the media between the electrodes can be matched for maximum electric power transfer. Further, the electrolysis gas may be generated at rates that enhance its residence time, while limiting the electrolysis gas “escape rate” out of the media and into the gas-filled headspace formed at the top of the bio-reactor.

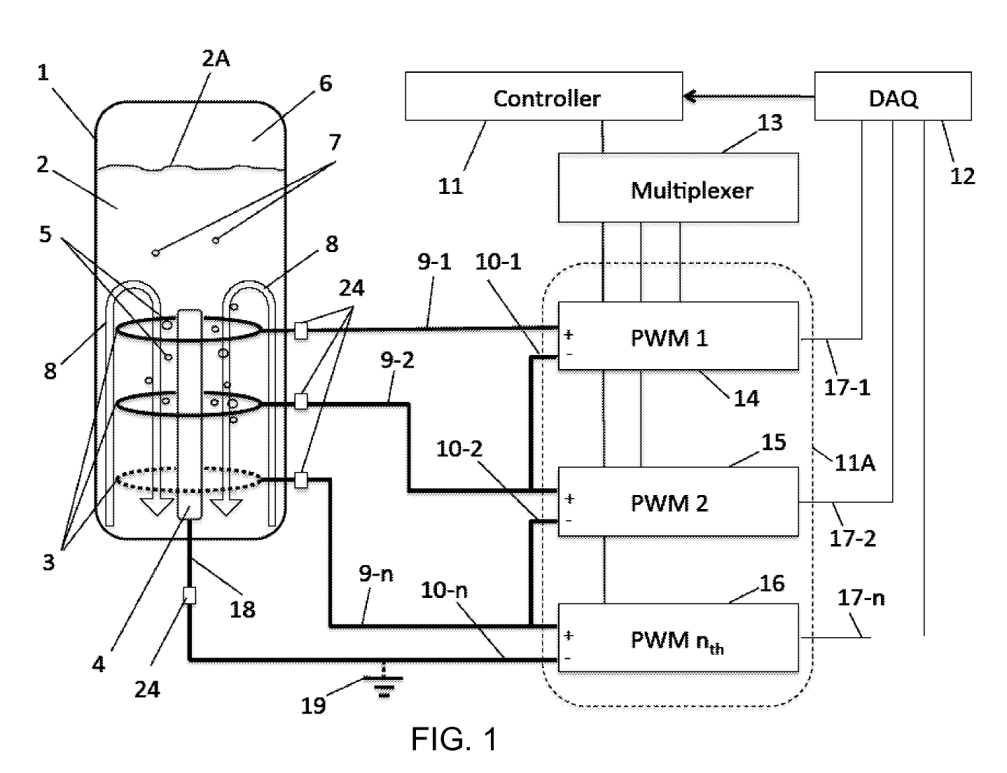

FIG. 1 illustrates a diagram of an example bio-electro reactor in accordance with embodiments of the present disclosure. The bio-electro reactor may generate electrolysis gas within a fermentor vessel 1 by controlling electric parameters by use of a programmable power box through a computer controller and user interface. Referring to FIG. 1, the bio-electro reactor includes the fermentor vessel 1 for holding a solution media 2. Ring-shaped electrodes 3 may be positioned within the vessel 1 and displaced from each other in a horizontal direction. The electrodes 3 may be suitably supported and electrically insulated by plugs connected to a sealing flange.

A central electrode 4 may be positioned within an interior of the electrodes 3. The central electrode 4 may be shaped as a cylinder with variable diameters to obtain different electrolysis gas results compatible with different types of solution media. The central electrode 4 may be formed by an electrode rod inserted and locked in position by a central electrically insulating and sealing plug, wherein said electrode rod is inserted and locked within a larger diameter cylindrically shaped cylinder whose surfaces are submerged and exposed to the solution media 2.

Each electrode 3 may be configured for series, parallel, or any other suitable combination of electrical connections 24 with a power box or electric source 11A formed by independent power supplies 14, 15, and 16. Power supplies 14, 15, and 16 may be controlled via an electrical controller, which may include a controller 11 and a data acquisition (DAQ) module 12. A suitable user interface may be controlled by a user for the setting and control of the power supplies 14, 15, and 16 for controlling frequency, voltage and current provided at electrodes 3. Power supplies 14, 15, and 16 may utilize pulse width modulators (PWM) or any other electronic power supply conditioning method and can detect or sense the electrical impedance represented by the solution media 2 as a dielectric transiting through electrodes 3 and 4. Real-time impedance information may be determined and communicated to the DAQ module 12. As impedance information is fed back to the DAQ module 12 and controller 11, electrical variables such as, but not limited to, current, voltage, and frequency may be adjusted so as to control the size of electrolysis gas bubbles 5. By controlling electrolysis gas size bubbles 5, the bio-electro reactor can control the buoyancy of the gas bubbles 5 and increase residence time in the solution media 2 and solubility. As electrolyte gases are produced, they tend to migrate toward the upper regions of vessel 1, as represented by bubbles 7 and can eventually escape from the solution surface 2A and collect in the solution-free, upper region 6 of vessel 1. To minimize this aspect, the controller 11 may be configured to set a sequenced activation/deactivation of electrodes 3. For example, the controller 11 may interface with a multiplexer 13 to vary frequency and duty cycle. As an example, the controller may include computer instructions for controlling the multiplexer 13.

In accordance with embodiments of the present disclosure, electrodes 3 may be configured to apply controlled frequencies and duty cycles via independent connections 24 to power supplies 14, 15, and 16. For example, FIG. 1 shows an electrical configuration in which each electrode 3 “sees” an increasing/decreasing potential from top to bottom so as to generate an electronic accelerator effect which, combined with multiplexed activation/deactivation of each individual electrode controlled by controller 11, exerts pumping forces on the solution 2 and generates recirculation flow patterns as indicated by arrows 8. In this manner, the electrolysis gases are forced to remain longer in the solution, thereby increasing their ability to dissolve in the solution 2. It is noted that solubility of electrolysis gases may also be enhanced by pressurizing vessel 1 while adjusting solution 2 temperature.

In accordance with embodiments of the present disclosure, the central electrode 4 may be connected to the lowest potential or ground 19 via line 18 and connection 24 while multiple electrodes 3 are activated and deactivated at various potentials to generate, in additional to electrolysis gases, forced flow circulation patterns in directions indicated by arrows 8. As each electrode 3 and 4 is electrically independent, their configurations can be changed to maximize impedance matching between the power supplies 14, 15, and 16 and the solution 2 at the conditions manifested between the central electrode 4 and electrodes 3. Electrodes 3 may be connected to power supplies 14, 15, and 16 via lines 9-1, 9-2, 9-n, 10-1, 10-2, and 10-n. Power supplies 14, 15, and 16 may be connected to the DAQ module 12 via lines 17-1, 17-2, and 17-n.

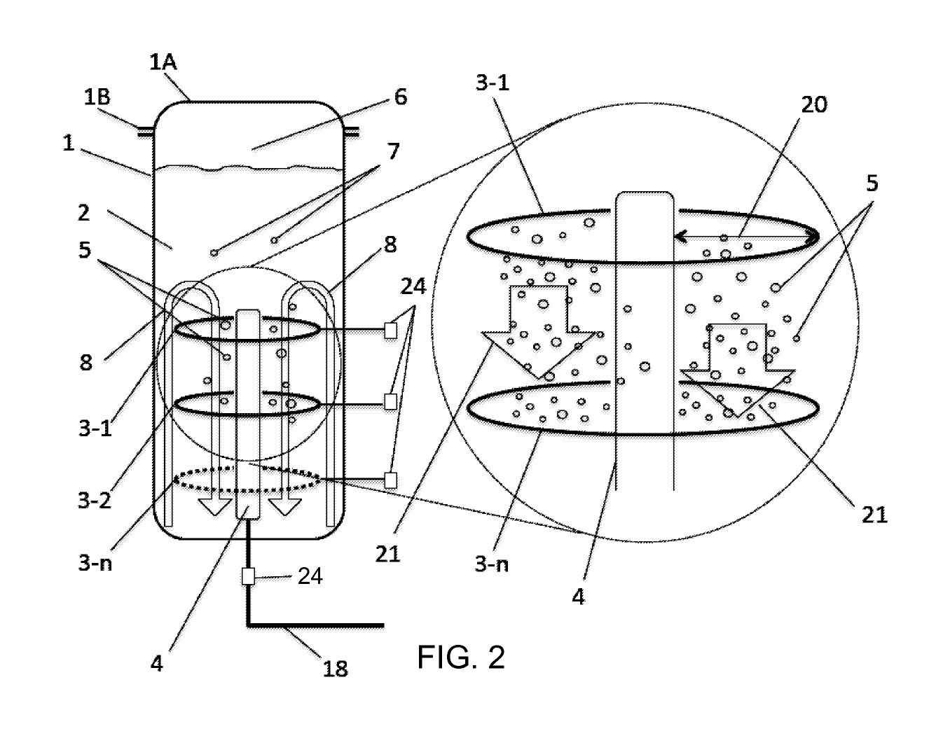

FIG. 2 depicts an example method of forcing flow circulation with a fermentor vessel in accordance with embodiments of the present disclosure. This figure shows details about electrolysis gas bubble formation and electronic pumping toward desired areas of the solution within the fermentor vessel. FIG. 2 shows the vessel 1, solution 2, and electrodes 3-1, 3-2, 3-n, and 4 shown in FIG. 1. Referring to FIG. 2, arrows 8 show flow patterns that can be generated by the bio-electro reactor without mechanical stirrers. The flow patterns can be generated by sampling the impedance formed in the solution 2 forming a dielectric at a distance 20 from the surfaces of anode and cathode electrodes 3-1, 3-2, 3-n and 4. As the electrical connections to power supplies 14, 15, and 16 are independent, the anode(s) may be configured to operate as cathode(s) and vice versa. As electrolysis gases bubbles 5 form between the electrodes 3-1 and 3-n as shown in the magnified representation of FIG. 2, the electrical impedance between the electrodes changes, and the power supplies 14, 15, and 16 may be adjusted accordingly to compensate for such changes in compliance with current, voltage and frequency parameters. Such adjustment may be dictated by control input of an operator. As the electrolysis gas bubbles 5 are ionized, by imparting a controlled electrostatic field exerted by electrodes 3-1, 3-2 and 3-n, bubbles 5 undergo controlled electrostatic forces so as to drive them toward desired areas of the solution 2. As shown by arrows 21 that indicate flow directions 21. For example, bubbles 5 are driven downward against buoyancy forces. The terminology adopted for electrode 3-n indicates the ability of adopting multiple or “n” electrodes. A lower portion and a top portion 1A of the vessel 1 may be connected together at a sealing flange 1B.

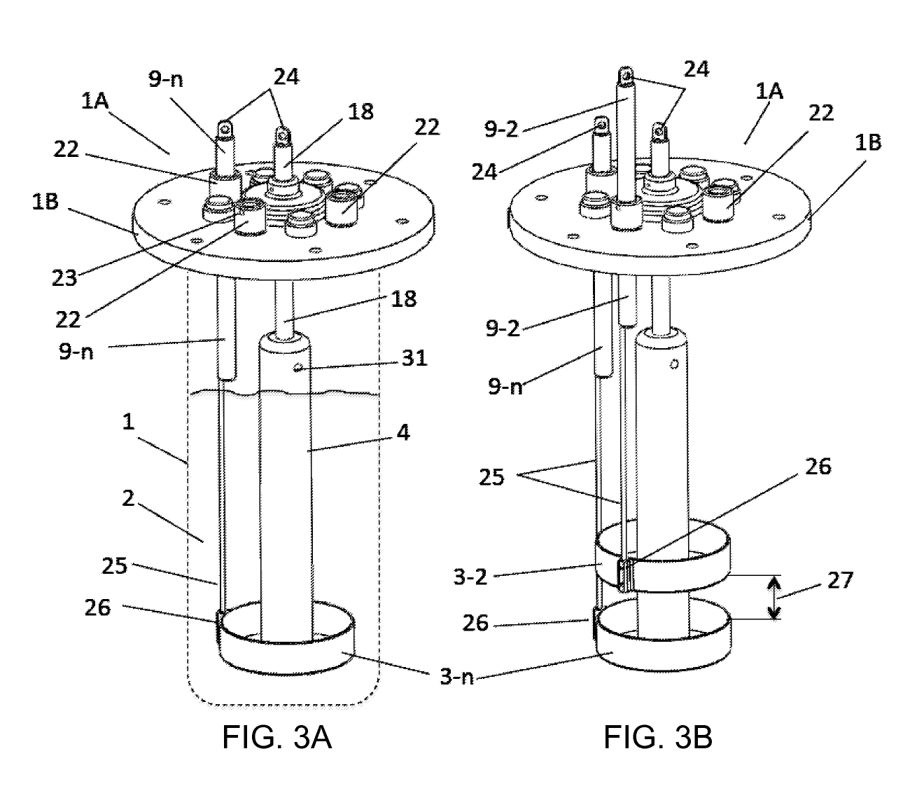

FIG. 3A illustrates a perspective view of assembled components of a fermentor vessel 1, electrodes 3-n and 4, and other assembly components in accordance with embodiments of the present disclosure. In this example, these components are non-invasively and reversibly retrofitted to a suitable bio-electro reactor. Flange 1B may be fitted with electrically-insulating and pressure-sealing plugs 22, which may be equipped with a locking mechanism 23 so as to allow sliding and locking of electrode rod 9-n at a desired elevation within the vessel 1. Electrode rod 9-n supports and electrically connects annular electrode 3-n by means of a mechanical coupler 26 and a conducting rod 25. The surface of conducting rod 25 may be electrically insulated. A locking mechanism 31 may lock electrode 4 at a desired elevation within the vessel 1. In this manner, a complete concentric cathode-anode electrode pair is formed between the electrode 3-n and the central electrode 4.

FIG. 3B illustrates another perspective view of the assembled component shown in FIG. 3A along with electrode 3-2 and its associated assembly components. Referring to FIG. 3B, two cathode-anode electrode pairs are formed by assembling electrodes 3-n and 3-2 with central electrode 4. This figure shows that the electrodes 3-2 and 3-n are spaced apart by a distance 27. The distance 27 may be adjusted to satisfy solution impedance and electrolysis gas production requirements as described herein.

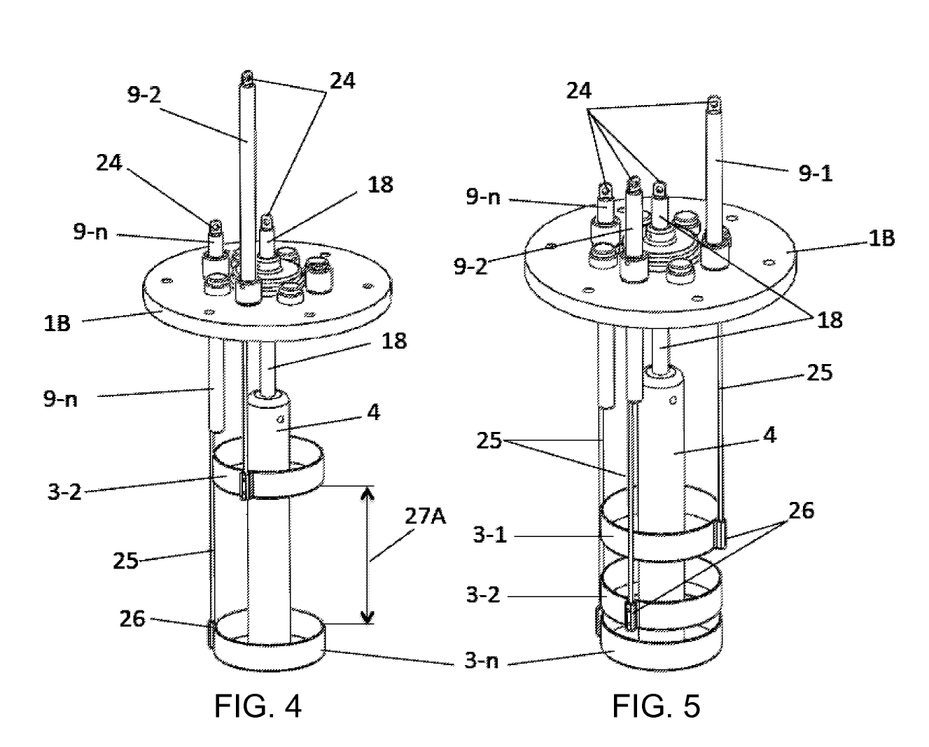

FIG. 4 illustrates a perspective view of the assembly shown in FIG. 3B with an extended distance 27A between the two annular electrodes 3-n and 3-2. As the electrodes are independent and electrically insulated from one another and flange 1B, outputs of lines 9-n and 10-n and 9-2 and 10-2 (shown in FIG. 1) may be configured in series or parallel. If the configuration is parallel, the annular electrodes may be used as electrical connection and mechanical support for screens or larger surface electrodes “wrapped” around electrodes 3-n and 3-2.

FIG. 5 illustrates a perspective view of the assembled component shown in FIG. 4 along with electrodes 3-1 and 3-2 and their associated assembly components. Referring to FIG. 5, the bio-electro reactor has three anode-cathode electrode pairs positioned at desired distances to match various solution 2 impedance characteristics at different elevations in the vessel.

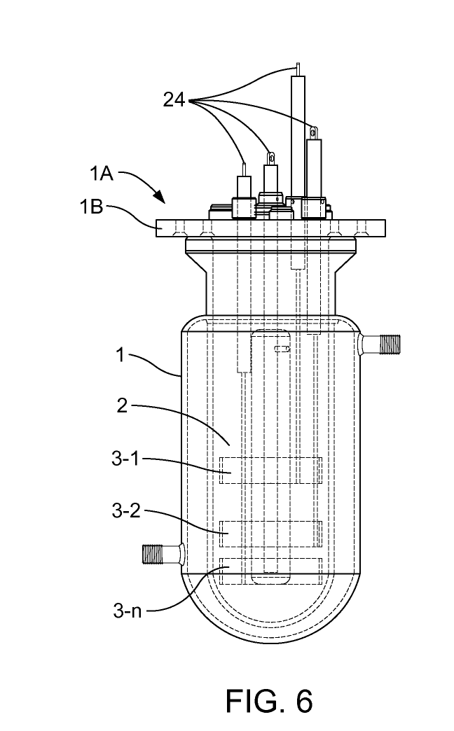

FIG. 6 illustrates a side view of a bio-reactor vessel 1 and top flange 1B. Referring to FIG. 6, three independent anode-cathode electrode pairs 3-1, 3-2, and 3-n are positioned within an interior space of the vessel 1.

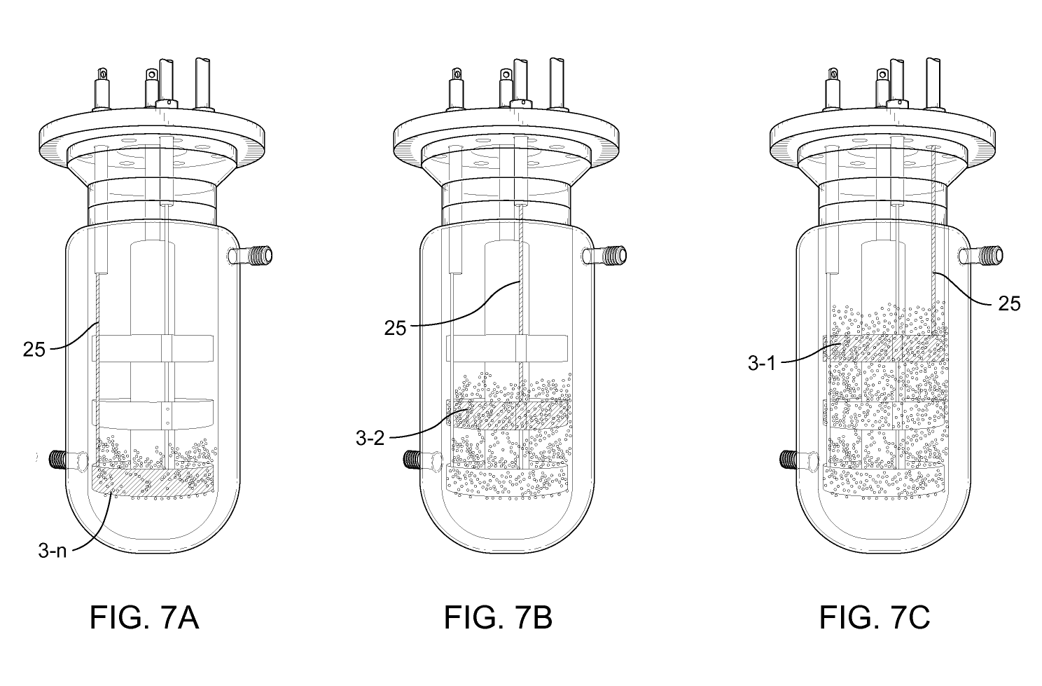

FIGS. 7A, 7B, and 7C illustrate perspective views showing sequential activation and deactivation of the annular electrodes 3-n, 3-2, and 3-1 to induce flow circulation patterns in accordance with embodiments of the present disclosure. The electrodes are maintained at optimized potentials without requiring electronic quenching. The sequence shown in these figures is only representative as all electrodes can be driven independently according to multiple configurations and activation and deactivation permutations. Referring to FIG. 7A, electrode 3-n may be activated while the other electrodes are deactivated to generate bubbles within a lower portion of the solution. Subsequently, as shown in FIG. 7B, electrode 3-2 may be activated while the other electrodes are deactivated to generate bubbles as depicted. Subsequent to activation of electrodes 3-n and 3-2, electrode 3-1 may be activated while the other electrodes are deactivated to generate bubbles as depicted.

The electrodes 3-1, 3-2, and 3-n may be separately activated and deactivated by an electric source, such as electric source 11A shown in FIG. 1. The electric source may apply voltages across the electrodes at different times with respect to each other. Further, the electric source may apply the voltage across the electrodes at a predetermined frequency. The electrical controller may include a DAQ module, controller, and multiplexer, such as the DAQ module 12, the controller 11, and the multiplexer 13 shown in FIG. 1.

An electrical controller may control the electric source to vary the voltage applied across the electrodes based on determined electrical impedance at the electrodes. For example, the power supplies 14, 15, and 16 shown in FIG. 1 may be configured to detect current flow through the solution or any biologically active substance and one or more electrodes. Alternatively, the power supplies 14, 15, and 16 may function as a detector for detecting any other electrical characteristic at one or more of the electrodes. The DAQ module 12 may receive a signal representing the electrical impedance, condition the signal, and communicate corresponding data to the controller 11. The controller 11 may determine a control input for the electric source 11A for generating a predetermined electric field within the interior of the vessel 1. The controller 11 may communicate the control input to the electric source 11A. Subsequently, the electric source 11A may apply voltage across the electrodes based on the control input.

In accordance with embodiments, a computing device (e.g., desktop computer, laptop computer, and the like) may be communicatively connected to an electric source, such as electric source 11A, for receiving signals therefrom. The computing device may store instructions in its memory for controlling the electric source to activate and deactivate applied voltages across the electrodes in a predetermined sequence. Further, the computing device may communicate control input to the electric source based on the stored instructions. In an example, the computing device may receive data representative of a current flow or any other electrical characteristic at one or more of the electrodes, and present a representation of the data to a user via a user interface, such as a display. A display of the computing device may be controlled to present a representation of the electrical characteristic. The electrical characteristic may be, for example, a voltage level, a current level, a frequency, and the like.

The programmable activation/deactivation of selected electrodes allows for the size of electrolysis gas bubbles to be controlled so as to maintain a bubble size corresponding to optimum buoyancy characteristics to enhance electrolysis gas residence time and dissolution. Additionally, the ability to control the activation/deactivation of the electrodes and electrical parameters to the electrodes can improve the flexibility of the system, allowing it to be used with a variety for growth mechanisms. As the total amount of electrolysis gases needed may be dictated by the requirements of the microbe of choice and its metabolic status (growth stage), the ability to tune the production of electrolysis gas may greatly enhance the efficiency of the fermentor.

In accordance with embodiments, the vessel may be flange-sealed for controlling temperature and pressure. The vessel may contain any of various culture media. The flange may be configured with electrically insulating and sealing plugs through which multiple electrodes may be positioned within the vessel. The electrically insulating and sealing plugs may be configured to lock in place the electrodes while electrically connecting the electrodes to the culture media on one side and to the programmable power supplies on the other side.

The central electrode rod and the multiple ring or annular band electrode rods allow on one side electrical connection with a power box wherein independent programmable power supplies provide electrical current to all electrodes according to preset electrode activation and deactivation sequences.

In an example application, a bio-electro reactor as disclosed herein may be used for the autotrophic growth of microbes. For example, a reactor may be used for the growth of Ralstonia eutropha. This Gram-negative soil bacterium has the ability to grow in aerobic and anaerobic environments, specifically utilizing hydrogen during its growth (Bowien and Schlegal, 1981). The fermentor may deliver the necessary nutrients for growth of Ralstonia in a safe manner. In an example, standard solution of trace nutrients are prepared (Schuster and Schlegal, 1967) and used to fill the fermentor. The nutrients may be referred to as the media. A central rod shaped electrode made of metal, such as a corrosion resistant metal such as stainless steel or metal coated with platinum, may be used as the anode. The cathode can be three ring-shaped electrodes made of, for example, metal such as stainless steel or metal coated with platinum. These electrically-isolated electrodes may be inserted into the media and connected to the controller. An external source of carbon dioxide may also be placed into the fermentor through a gas sparing system. The controller may be turned on to pulse the voltage on each individual electrode from between 0.8 volts to 2.4 volts for seconds each. Subsequently, small bubbles of hydrogen and oxygen appear in the solution. The fermentor may then have nutrient necessary to grow the Ralstonia, hydrogen, oxygen, carbon dioxide, and trace nutrients in the media. The microbe is allowed to grow until the concentration of the desired product is achieved.

In accordance with embodiments, a vessel may contain a biologically active substance. For example, the biologically active may be a microbe. The microbe is classified as a chemoautotrophic microbe, which may be eubacteria. The eubacteria may be, for example, Ralstonia eutropha H16 or a derivative thereof. The strain of Ralstonia eutropha has been genetically engineered to biosynthesize and secrete a mixture of hydrocarbons and the like. The mixture of hydrocarbons may include methyl ketones. In an example, the methyl ketones may include 2-undecanone, 2-tridecanone, and 2-pentadecanone. The genes A0459-0464 and A1526-1531 encoding the two beta-oxidation pathways may be deleted from the Ralstonia eutropha H16 chromosome to create a R. eutropha Δ(A0459-0464, A1526-1531) strain. R. eutropha Δ(A0459-0464, A1526-1531) may contain the plasmid pMKP expressing the E. coli TesA protein, the E. coli FadB and FadM proteins and the Micrococcus luteus Mlut_{—}11700 gene encoding an acyl-CoA dehydrogenase. R. eutropha Δ(A0459-0464, A1526-1531) may express the E. coli TesA, FadB and FadM proteins and the Micrococcus luteus Mlut_{—}11700 gene from the R. eutropha Δ(A0459-0464, A1526-1531) chromosome. The biologically active substance comprises a liquid growth medium comprising sodium phosphate dibasic, potassium sulfate monobasic, sodium bicarbonate, ammonium sulfate, ammonium iron (II) citrate, nickel sulfate, calcium sulfate, and magnesium sulfate.

In accordance with embodiments, a bio-electro reactor vessel may be at least partially filled with growth medium. The production strain is inoculated into the vessel containing growth medium at a density sufficient for growth of the microbe. In an example, the production strain is inoculated into the fermenter containing growth medium at a density from 0.1 to 10% (v/v). In another example, the production strain is inoculated into the vessel containing growth medium at a density of 1% (v/v). The vessel containing the production strain and the growth medium is incubated between 20° C. and 40° C. In an example, the vessel containing the production strain and the growth medium is incubated at 30° C. In an example, a solution of arabinose is added to the bio-electro reactor containing the production strain and the growth medium. A solution of 0.01% to 1% arabinose (w/v) may be added to the bio-electro reactor containing the production strain and the growth medium. In an example, a solution of about 0.2% arabinose (w/v) is added to the vessel containing the production strain and the growth medium. An immiscible solvent, such as decane, may be added to the vessel containing the production strain, the growth medium, or a solution of arabinose. In an example, 5-20% decane (v/v) is added to the bio-electro reactor containing the production strain, the growth medium, or a solution of arabinose. In another example, about 10% decane (v/v) is added to the bio-electro reactor containing the production strain, the growth medium, or a solution of arabinose.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is noted that the components and techniques disclosed herein may be suitably utilized with any suitable bio-electro reactor regardless of their size as the electrodes can be scaled and adapted to fit the reactor vessel.

The various techniques described herein may be implemented with hardware or software or, where appropriate, with a combination of both. Thus, the methods and apparatus of the disclosed embodiments, or certain aspects or portions thereof, may take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for practicing the presently disclosed subject matter. In the case of program code execution on programmable computers, the computer will generally include a processor, a storage medium readable by the processor (including volatile and non-volatile memory and/or storage elements), at least one input device and at least one output device. One or more programs may be implemented in a high level procedural or object oriented programming language to communicate with a computer system. However, the program(s) can be implemented in assembly or machine language, if desired. In any case, the language may be a compiled or interpreted language, and combined with hardware implementations.

The described methods and apparatus may also be embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, a video recorder or the like, the machine becomes an apparatus for practicing the presently disclosed subject matter. When implemented on a general-purpose processor, the program code combines with the processor to provide a unique apparatus that operates to perform the processing of the presently disclosed subject matter.

Features from one embodiment or aspect may be combined with features from any other embodiment or aspect in any appropriate combination. For example, any individual or collective features of method aspects or embodiments may be applied to apparatus, system, product, or component aspects of embodiments and vice versa.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. A bio-electro reactor for control of electrolysis gases bubbles within a biologically-active substance, the bio-electro reactor comprising:
a vessel defining an interior for holding a biologically-active substance;
a plurality of electrodes positioned to be electrically coupled with at least a portion of the biologically-active substance;
an electric source configured to apply voltage across the plurality of electrodes; and
an electrical controller configured to determine an electrical impedance at one or more of the electrodes for use in controlling electrolysis gases bubbles within the biologically active substance.

2. The bio-electro reactor of claim 1, wherein the electrodes are one of ring-shaped, screen-shaped, and rectangular-shaped.

3. The bio-electro reactor of claim 1, wherein the electrodes are displaced from each other.

4. The bio-electro reactor of claim 1, wherein the electric source is configured to apply different voltages across the plurality of electrodes.

5. The bio-electro reactor of claim 1, wherein the electric source is configured to apply different voltages across the plurality of electrodes at controlled frequencies and duty-cycle.

6. The bio-electro reactor of claim 1, wherein the electric source is configured to separately activate and deactivate the applied voltages across the plurality of electrodes at different times with respect to each other.

7. The bio-electro reactor of claim 1, wherein the electric source is configured to apply the voltage across the plurality of electrodes at a predetermined frequency.

8. The bio-electro reactor of claim 1, wherein the electrical controller is configured to vary the voltage applied across the plurality of electrodes based on the determined electrical impedance.

9. The bio-electro reactor of claim 1, wherein the electrical source is configured to apply voltage across the plurality of electrodes for controlling voltage across the biologically-active substance to thereby control a size and buoyancy of the electrolysis gases bubbles.

10. The bio-electro reactor of claim 9, wherein the electrical controller is configured to:
detect current flow through biologically active substance and at least one of the electrodes;
determine a control input for the electric source for generating a predetermined electrical field within the interior of the vessel; and
communicate the control input to the electric source,
wherein the electric source is configured to apply voltage across the plurality of electrodes based on the control input.

11. The bio-electro reactor of claim 1, further comprising a computing device communicatively connected to the electric source and configured to:
store instructions for controlling the electric source to activate and deactivate the applied voltages across the plurality of electrodes in a predetermined sequence; and
communicate control input to the electric source based on the stored instructions.

12. The bio-electro reactor of claim 1, further comprising:
a detector communicatively connected to at least one of the electrodes, and configured to detect an electrical characteristic at the at least one of the electrodes; and
a computing device communicatively connected to the detector, and configured to present a representation of the electrical characteristic to a user.

13. The bio-electro reactor of claim 12, wherein the computing device comprises a display configured to display a visual representation of the electrical characteristic.

14. The bio-electro reactor of claim 12, wherein the electrical characteristic is one of a voltage level, a current level, and a frequency.

15. The bio-electro reactor of claim 1, wherein the electric source is configured to output a data signal representative of the applied voltage of the electric source; and
a computing device communicatively connected to the electric source for receiving the output data signal, and configured to present a representation of the output data signal to a user.

16. The bio-electro reactor of claim 15, wherein the computing device comprises a display configured to display a visual representation of the output data signal.

17. The bio-electro reactor of claim 15, wherein the output data signal is one of a voltage level and a frequency of the applied voltage across the plurality of electrodes.

18. The bio-electro reactor of claim 1, wherein application of the voltage across the plurality of electrodes causes manifestation of voltage, frequency, and current across the biologically-active substance.

19. The bio-electro reactor of claim 1, wherein the electrical controller is in electrical communication with the plurality of electrodes, configured to receive electrical signals from the electrodes, and configured to determine an electric impedance of the biologically-active substance based on the electrical signals.

20. The bio-electro reactor of claim 19, wherein the electrical controller is configured to independently control each of the electrodes based on the determined electric impedance of the biologically-active substance.

21. The bio-electro reactor of claim 19, wherein the electrical controller is configured to independently vary at least one of a voltage, current, frequency, and duty-cycle of each of the electrodes based on the determined electric impedance of the biologically-active substance.

22. A method of using a bio-electro reactor for control of electrolysis gases bubbles within a biologically-active substance, the method comprising:
providing a bio-electro reactor comprising:
a vessel defining an interior for holding a biologically-active substance; and
a plurality of electrodes positioned to be electrically coupled with at least a portion of the biological-active substance; and
applying voltage between the interior electrode and each of the exterior electrodes.

23. The method of claim 22, wherein the electrodes are ring-shaped.

24. The method of claim 22, wherein the electrodes are displaced from each other.

25. The method of claim 22, wherein applying voltage comprises applying different voltages across the plurality of electrodes.

26. The method of claim 22, further comprising applying different voltages across the plurality of electrodes at controlled frequencies and duty-cycle.

27. The method of claim 22, further comprising separately activating and deactivating the applied voltages across the plurality of electrodes at different times with respect to each other.

28. The method of claim 22, wherein applying voltage comprises applying the voltage across the plurality of electrodes at a predetermined frequency.

29. The method of claim 22, further comprising varying the voltage across the plurality of electrodes based on the determined electrical impedance.

30. The method of claim 22, further comprising applying voltage across the plurality of electrodes for controlling voltage across the biologically-active substance to thereby control a size and buoyancy of the electrolysis gases bubbles.

31. The method of claim 30, further comprising detecting current flow through at least one of the electrodes, and
at a computing device:
determining a control input for the electric source for generating a predetermined electrical field within the interior of the vessel; and
communicating the control input to the electric source,
wherein the electric source is configured to apply voltage across the plurality of electrodes based on the control input.

32. The method of claim 22, further comprising:
storing instructions for controlling the electric source to activate and deactivate the applied voltages across the plurality of electrodes in a predetermined sequence; and
communicating control input to the electric source based on the stored instructions.

33. The method of claim 22, further comprising:
detecting an electrical characteristic at the at least one of the electrodes; and
presenting a representation of the electrical characteristic to a user.

34. The method of claim 33, further comprising displaying a visual representation of the electrical characteristic.

35. The method of claim 33, wherein the electrical characteristic is one of a voltage level, a current level, and a frequency.

36. The method of claim 22, further comprising generating a data signal representative of the applied voltage of the electric source; and
at a computing device:
receiving the output data signal; and
presenting a representation of the output data signal to a user.

37. The method of claim 36, further comprising displaying a visual representation of the output data signal.

38. The method of claim 36, wherein the output data signal is one of a voltage level and a frequency of the applied voltage across the plurality of electrodes.

39. The method of claim 22, wherein application of the voltage across the plurality of electrodes causes manifestation of voltage, frequency, and current across the biologically-active substance.

40. The method of claim 22, further comprising:
receiving electrical signals from the electrodes; and
determining an electric impedance of the biologically-active substance based on the electrical signals.

41. The method of claim 40, further comprising independently controlling each of the electrodes based on the determined electric impedance of the biologically-active substance.

42. The method of claim 40, further comprising independently varying at least one of a voltage, current, frequency, and duty-cycle of each of the electrodes based on the determined electric impedance of the biologically-active substance.

43. A bio-electro reactor for growth of microbes for production of chemicals via control of electrolysis gases bubbles within a biologically-active substance, the bio-electro reactor comprising:
a vessel defining an interior for holding a biologically-active substance;
a plurality of electrodes positioned to be electrically coupled with at least a portion of the biologically-active substance;
an electric source configured to apply voltage across the plurality of electrodes; and
an electrical controller configured to determine an electrical impedance at one or more of the electrodes for use in controlling electrolysis gases bubbles within the biologically active substance.

44. The bio-electro reactor of claim 43, wherein the biologically active substance comprises a microbe.

45. The bio-electro reactor of claim 43, wherein the microbe is classified as a chemoautotrophic microbe.

46. The bio-electro reactor of claim 45, wherein the chemoautotrophic microbe is a eubacteria.

47. The bio-electro reactor of claim 46, wherein the eubacteria is Ralstonia eutropha or a derivative thereof.

48. The bio-electro reactor of claim 47, wherein the strain of Ralstonia eutropha has been genetically engineered to biosynthesize and secrete a mixture of hydrocarbons.

49. The bio-electro reactor of claim 48, wherein the mixture of hydrocarbons comprises methyl ketones.

50. The bio-electro reactor of claim 49, wherein the mixture of methyl ketones comprises 2-undecanone, 2-tridecanone, and 2-pentadecanone.

51. The bio-electro reactor of claim 50, wherein the genes A0459-0464 and A1526-1531 encoding the two beta-oxidation pathways are deleted from a Ralstonia eutropha H16 chromosome to create a R. eutropha Δ(A0459-0464, A1526-1531) strain.

52. The bio-electro reactor of claim 51, wherein R. eutropha Δ(A0459-0464, A1526-1531) contains the plasmid pMKP expressing the E. coli TesA protein, the E. coli FadB and FadM proteins and the Micrococcus luteus Mlut_{—}11700 gene encoding an acyl-CoA dehydrogenase.

53. The bio-electro reactor of claim 52, wherein R. eutropha Δ(A0459-0464, A1526-1531) expresses the E. coli TesA, FadB and FadM proteins and the Micrococcus luteus Mlut_{—}11700 gene from the R. eutropha Δ(A0459-0464, A1526-1531) chromosome.

54. The bio-electro reactor of claim 43, wherein the biologically active substance comprises a liquid growth medium comprising sodium phosphate dibasic, potassium sulfate monobasic, sodium bicarbonate, ammonium sulfate, ammonium iron (II) citrate, nickel sulfate, calcium sulfate, and magnesium sulfate.

55. The bio-electro reactor of claim 43, wherein the vessel is at least partially filled with growth medium.

56. The bio-electro reactor of claim 43, wherein the production strain is inoculated into the vessel containing growth medium at a density sufficient for growth of the microbe.

57. The bio-electro reactor of claim 43, wherein the production strain is inoculated into the fermenter containing growth medium at a density from 0.1 to 10% (v/v).

58. The bio-electro reactor of claim 43, wherein the production strain is inoculated into the vessel containing growth medium at a density of 1% (v/v).

59. The bio-electro reactor of claim 43, wherein the vessel containing the production strain and the growth medium is incubated between 20° C. and 40° C.

60. The bio-electro reactor of claim 43, wherein the vessel containing the production strain and the growth medium is incubated at 30° C.

61. The bio-electro reactor of claim 43, wherein the vessel containing the production strain and the growth medium is incubated between 30° C.

62. The bio-electro reactor of claim 43, wherein a solution of arabinose is added to the bio-electro reactor containing the production strain and the growth medium.

63. The bio-electro reactor of claim 43, wherein a solution of 0.01% to 1% arabinose (w/v) is added to the bio-electro reactor containing the production strain and the growth medium.

64. The bio-electro reactor of claim 43, wherein a solution of about 0.2% arabinose (w/v) is added to the vessel containing the production strain and the growth medium.

65. The bio-electro reactor of claim 43, wherein decane is added to the vessel containing the production strain, the growth medium, or a solution of arabinose.

66. The bio-electro reactor of claim 43, wherein 5-20% decane (v/v) is added to the bio-electro reactor containing the production strain, the growth medium, or a solution of arabinose.

67. The bio-electro reactor of claim 43, wherein about 10% decane (v/v) is added to the bio-electro reactor containing the production strain, the growth medium, or a solution of arabinose.

68. A method of using a bio-electro reactor for growth of microbes for production of chemicals via control of electrolysis gases bubbles within a biologically-active substance, the method comprising:
providing a bio-electro reactor comprising:
a vessel defining an interior for holding a biologically-active substance; and
a plurality of electrodes positioned to be electrically coupled with at least a portion of the biologically-active substance;
applying voltage across the plurality of electrodes; and
determining an electrical impedance at one or more of the electrodes for use in controlling electrolysis gases bubbles within the biologically active substance.

69. The method of claim 68, wherein the biologically active substance comprises a microbe.

70. The method of claim 68, wherein the microbe is classified as a chemoautotrophic microbe.

71. The method of claim 70, wherein the chemoautotrophic microbe is a eubacteria.

72. The method of claim 71, wherein the eubacteria is Ralstonia eutropha or a derivative thereof.

73. The method of claim 72, wherein the strain of Ralstonia eutropha has been genetically engineered to biosynthesize and secrete a mixture of hydrocarbons.

74. The method of claim 73, wherein the mixture of hydrocarbons comprises methyl ketones.

75. The method of claim 74, wherein the mixture of methyl ketones comprises 2-undecanone, 2-tridecanone, and 2-pentadecanone.

76. The method of claim 75, wherein the genes A0459-0464 and A1526-1531 encoding the two beta-oxidation pathways are deleted from the Ralstonia eutropha H16 chromosome to create a R. eutropha Δ(A0459-0464, A1526-1531) strain.

77. The method of claim 76, wherein R. eutropha Δ(A0459-0464, A1526-1531) contains the plasmid pMKP expressing the E. coli TesA protein, the E. coli FadB and FadM proteins and the Micrococcus luteus Mlut_{—}11700 gene encoding an acyl-CoA dehydrogenase.

78. The method of claim 77, wherein R. eutropha Δ(A0459-0464, A1526-1531) expresses the E. coli TesA, FadB and FadM proteins and the Micrococcus luteus Mlut_{—}11700 gene from the R. eutropha Δ(A0459-0464, A1526-1531) chromosome.

79. The method of claim 68, wherein the biologically active substance comprises a liquid growth medium comprising sodium phosphate dibasic, potassium sulfate monobasic, sodium bicarbonate, ammonium sulfate, ammonium iron (II) citrate, nickel sulfate, calcium sulfate, and magnesium sulfate.

80. The method of claim 68, wherein the vessel is at least partially filled with growth medium.

81. The method of claim 68, wherein the production strain is inoculated into the vessel containing growth medium at a density sufficient for growth of the microbe.

82. The method of claim 68, wherein the production strain is inoculated into the fermenter containing growth medium at a density from 0.1 to 10% (v/v).

83. The method of claim 68, wherein the production strain is inoculated into the vessel containing growth medium at a density of 1% (v/v).

84. The method of claim 68, wherein the vessel containing the production strain and the growth medium is incubated between 20° C. and 40° C.

85. The method of claim 68, wherein the vessel containing the production strain and the growth medium is incubated at 30° C.

86. The method of claim 68, wherein the vessel containing the production strain and the growth medium is incubated between 30° C.

87. The method of claim 68, wherein a solution of arabinose is added to the bio-electro reactor containing the production strain and the growth medium.

88. The method of claim 68, wherein a solution of 0.01% to 1% arabinose (w/v) is added to the bio-electro reactor containing the production strain and the growth medium.

89. The method of claim 68, wherein a solution of about 0.2% arabinose (w/v) is added to the vessel containing the production strain and the growth medium.

90. The method of claim 68, wherein decane is added to the vessel containing the production strain, the growth medium, or a solution of arabinose.

91. The method of claim 68, wherein 5-20% decane (v/v) is added to the bio-electro reactor containing the production strain, the growth medium, or a solution of arabinose.

92. The method of claim 68, wherein about 10% decane (v/v) is added to the bio-electro reactor containing the production strain, the growth medium, or a solution of arabinose.
